(19) United States
(12) Patent Application Publication
(10) Pub. No.: US 2025/0256037 A1
(43) Pub. Date: Aug. 14, 2025

(54) AUTO-INJECTOR WITH VISUAL INDICATION

(71) Applicant: Owen Mumford Limited, Woodstock, Oxfordshire (GB)

(72) Inventor: Robert William BRUNS, Woodstock, Oxfordshire (GB)

(21) Appl. No.: 18/849,336

(22) PCT Filed: Mar. 27, 2023
(86) PCT No.: PCT/EP2023/057767

§ 371 Date: Sep. 20, 2024

(30) Foreign Application Priority Data
Mar. 28, 2022 (GB) 2204308.7

(51) Int. Cl. A61M 5/315; A61M 5/20; A61M 5/31

(52) U.S. Cl. CPC A61M 5/31568; A61M 5/2033; A61M 2005/2013; A61M 2005/3126; A61M 2205/3317; A61M 2205/3327; A61M 2205/3569; A61M 2205/505

(57) ABSTRACT

The present invention relates to an auto-injector. The auto-injector includes a housing for receiving a syringe having a plunger, a needle, and a barrel containing a dose of medicament; a drive mechanism configured to, when triggered by a user, automatically drive the plunger from a first position to a second position to expel the dose from the barrel of the syringe through the needle at a front end of the auto-injector; a detector arrangement configured to detect a state of the plunger including at least departure from the first position and arrival at the second position; and an indicator arrangement comprising a display having one or more electrically-operated display elements and configured to provide, responsive to the detector arrangement detecting the departure of the plunger from the first position, a visual indication comprising cycles of indications, the cycles of indications repeating until the detector arrangement detects the arrival of the plunger at the second position.

### TECHNICAL FIELD

The present disclosure relates to an auto-injector for delivering a medicament from a syringe. In particular, the disclosure relates to an auto-injector which increases the likelihood of optimal and complete full-dose drug delivery.

### BACKGROUND

Injection devices, in particular auto-injectors, are commonly used to self-administer injections of medicament. Such auto-injectors are configured to receive a syringe containing the medicament in a barrel, and then drive a plunger of the syringe into the barrel in order to deliver the medicament. Typically, the driving of the plunger occurs without the need for any driving force to be applied by the user, other than perhaps a small amount of force to trigger the auto-injector.

During the delivery of a medicament from an auto-injector, the user is required to hold a front end of the auto-injector against the injection site in order to keep the needle penetrated in the injection site, while the medicament is delivered. Specifically, the user may be required to hold the needle in place while the plunger moves completely through the barrel, leading to a so-called ‘full-dose delivery’ of the medicament. In order to aid the user in this process, an auto-injector may be provided with an indication device that generates an indication corresponding to the stage of use of the device.

However, various factors can lead to a sub-optimal, or incomplete, delivery of medicament, despite the user holding the auto-injector against the injection site until the plunger has moved completely through the barrel.

An improved auto-injector is required to ensure optimal and complete delivery of the medicament.

### SUMMARY

In accordance with a first aspect, there is provided an auto-injector according to claim 1.

The auto-injector comprises a housing for receiving a syringe having a plunger, a needle, and a barrel containing a dose of medicament. The auto-injector further comprises a drive mechanism configured to, when triggered by a user, automatically drive the plunger from a first position to a second position to expel the dose from the barrel of the syringe through the needle at a front end of the auto-injector. The auto-injector further comprises a detector arrangement configured to detect a state of the plunger including at least departure from the first position and arrival at the second position. The auto-injector further comprises an indicator arrangement. The indicator arrangement may comprise a display having one or more electrically-operated display elements. The indicator arrangement may be configured to provide, responsive to the detector arrangement detecting the departure of the plunger from the first position, a visual indication comprising cycles of indications. The cycles of indications may repeat until the detector arrangement detects the arrival of the plunger at the second position.

Advantageously, the provision of a visual indication informs a user that the delivery sequence has begun. This serves to show the user that the device is functioning correctly and that delivery is ongoing, and so the auto-injector should not be removed from the injection site yet. Premature removal of the auto-injector can lead to sub-optimal or incomplete delivery, and so ensuring that the user is aware that the delivery is ongoing ensures that the delivery is optimal and complete. The visual indication may herein be referred to as a ‘progress indication’.

It is an appreciation of the Applicant that providing cycles of indication ensures that the user is aware that the delivery is actually ongoing, and not simply that the injector is active or awaiting further input. This is in contrast to, say, auto-injectors that provide static visual indications. Such auto-injectors may provide one or display elements that are active (e.g. illuminated) during delivery, but that do not change or repeat during delivery. This can be confusing for the user, as they are not aware of whether the injector is performing any function (e.g. delivery) or is simply indicating that it is switched on. By providing cycles of indication, the user is aware that a function, in this case delivery, is ongoing and so the user should wait for further indications before taking any further actions. The Applicant has found that this has an impact on the likelihood of the user prematurely removing the auto-injector from the injection site. Specifically, a user is less likely to prematurely remove the auto-injector if a repeating cycle of indications is used.

The term ‘cycle’ is used herein to describe repetition in time, i.e. repeated periods of indication, rather than necessarily a geometric cycle in a visual sense, i.e. motion in a circle.

The indicator arrangement may additionally or alternatively be configured to provide a cyclical indication in a manner other than a visual indication. For example, the indicator arrangement may be configured to provide, responsive to the detector arrangement detecting the departure of the plunger from the first position, a haptic indication comprising cycles of indications. Alternatively or additionally, the indicator arrangement may be configured to provide, responsive to the detector arrangement detecting the departure of the plunger from the first position, an audible indication comprising cycles of indications.

The electrically-operated display elements may comprise an illumination device as described herein. For example, the electrically-operated display elements may comprise one or more light emitting diodes (LEDs) and/or a display screen. Alternatively or additionally, the electrically-operated display elements may comprise a liquid-crystal display (LCD). In embodiments in which the electrically-operated display elements comprise a display screen, any of the visual indications described herein may be provided in the form of an animation on the display screen.

The indicator arrangement may further comprise one or more auxiliary indicators. The auxiliary indicators may be configured to provide an indication of one or more states of the auto-injector that may be unrelated to the delivery sequence. For example, the auxiliary indicators may be configured to indicate one or more of the following: an power status of the auto-injector (e.g. whether the auto-injector is currently powered); a battery status of the auto-injector (e.g. an indication of the remaining battery life of the auto-injector, and/or whether the auto-injector is or requires recharging); a maintenance status of the auto-injector (e.g. whether the auto-injector requires repair, maintenance, and/or regular servicing); and a connectivity status of the auto-injector (e.g. whether the auto-injector is connected, connecting, disconnected, and/or disconnecting from another device, for example, via a short-range transmission protocol such as Bluetooth).

In some embodiments, the cycles of indications present a sequence that moves in a continuous or repeated direction.

In some embodiments, the one or more electrically-operated display elements are arranged in a circular arrangement.

It is understood that the term ‘circular’ in this context means that the arrangement is substantially visually cyclical. That is, the term ‘circular’ also encompasses other similar arrangements such as elliptical or oval arrangements.

Advantageously, a circular arrangement of display elements provides a visual indication that does not have a visual end point. This provides an implicit indication to the user that a process is ongoing, reducing the likelihood that the user will consider a process to have terminated while the indication is provided. This in turn assists the user in performing the injection to provide an optimal and complete delivery of medicament.

In some embodiments, the one or more electrically-operated display elements are configured to be operated sequentially. The sequential operation of the one or more electrically-operated display elements may give the appearance of rotation through the electrically-operated display elements.

In some embodiments, the one or more electrically-operated display elements are arranged in a linear arrangement.

In some embodiments, the one or more electrically-operated display elements are configured to be operated sequentially to give the appearance of scrolling through the electrically-operated display elements.

Alternatively, the one or more electrically-operated display elements may be arranged in an arrangement other than circular or linear. For example, the one or more electrically-operated display elements may be arranged in a different visually cyclical arrangement, such as a square or other polygonal shape.

Alternatively, the one or more electrically-operated display elements may be configured to provide cycles of indications based on changes of brightness or colour. For example, the one or more electrically-operated display elements may be configured to repeatedly increase and decrease in brightness. Such a visual indication is sometimes referred to as a ‘breathing’ indication.

In some embodiments, the indicator arrangement is further configured to continue repeating the cycles of indication during a holding period that starts from when the detector arrangement detects the arrival of the plunger at the second position.

Advantageously, continuing the cycles during a holding period means that no change in indication is provided when the plunger's arrival at the second position is detected. This prevents the user from mistakenly considering that it is acceptable to remove the auto-injector once the plunger has reached the second position. In other words, if the cycles were to stop (or an alternative indication were to be provided) when the plunger arrives at the second location, the user may take this change in indication to mean that the injection process is complete. The user may then subsequently remove the auto-injector from the injection site before a complete and optimal dose is delivered (because either the bung has not expanded or the medicament has not dispersed into the soft tissue). By continuing the same indication during this time, the user is simply informed to continue holding the auto-injector, thereby allowing complete and optimal delivery to occur. The holding period may be compared and/or defined relative to the threshold time as described herein. For example, the holding period may be equal to or greater than the threshold time.

In some embodiments, the indicator arrangement is further configured to provide a completion indication notifying the user that the auto-injector can be removed from the injection site. The indicator arrangement may be configured to provide the completion indication responsive to the expiry of the holding period.

The completion indication may be provided by the one or more electrically-operated display elements. For example, the completion indication may comprise flashing of the electrically-operated display elements. Alternatively, the completion indication may simply be the termination of the cycles of indication. Alternatively or additionally, the completion indication may comprise an audible or haptic indication.

In some embodiments, the holding time is greater than or equal to a typical period of time taken for a resilient syringe bung to relax and expand.

This typical period may be referred to herein as a relaxation time. Advantageously, the holding period being longer than the relaxation time allows the bung to expand before the auto-injector is removed from the injection site. Since the expansion of the bung may result in more medicament being delivered from the syringe, this ensures that the full dose has been delivered.

In some embodiments, the holding time is greater than or equal to a typical period of time taken for a medicament to disperse into soft tissue.

This typical period may be referred to herein as a dispersion time. Advantageously, the holding period being longer than the dispersion time allows the medicament to disperse before the auto-injector is removed from the injection site. Since the medicament may leak if it has not sufficiently dispersed before the auto-injector is removed, this ensures that the medicament does not leak from the injection site.

The holding time may be customizable at the point of programming or manufacture of the auto-injector. This allows a manufacturer to define a suitable holding time based on known or accepted relaxation or dispersion times, for example, based on a particular medicament to be delivered by the auto-injector. Alternatively or additionally, the holding time may be customizable by the user, for example, using inputs on the auto-injector or in an application of a user device that is configured to communicate with the auto-injector. This allows the user to change the holding time before using the auto-injector to deliver medicament. For example, the user may change the holding time to suit the particular medicament being delivered, if the auto-injector is used to deliver multiple different medicaments.

In some embodiments, the detector arrangement comprises a first switch located at the first position. The first switch may be configured to detect a departure of the plunger from the first position.

Advantageously, detecting the departure of the plunger from a position has been found by the Applicant to be more reliable than detecting the arrival of the plunger at the position. The detection of the plunger arriving or passing a certain position requires the detection of movement of the plunger through that certain position. The movement of the plunger in a delivery stroke can be of relatively high speed. This means that, when detecting arrival, the detector must have an appropriately high sample rate in order to detect the high speed arrival or passing of the certain position. This can lead to reliability issues where known switches do not register the arrival or passing of the plunger because it moves at a speed that is too fast for the sampling rate of the detector. By detecting the departure, the plunger is already located at the certain position, and so the switch is already actuated. The switch therefore must only register its release when the plunger leaves. Such an arrangement allows for more accurate detection of the plunger's movement compared to the detection of arrival.

While the detection of departure is preferred, it is also contemplated that the first switch could be located at an intermediate position between the first position and the second position, optionally nearer to the first position than the second position. In such a case, the first switch would be configured to detect the arrival of the plunger at the intermediate position as a proxy for its departure from the first position.

In alternative embodiments, the detector arrangement may be configured to continuously detect the position of the plunger. For example, the detector arrangement may comprise a rotary or linear encoder for detecting the position of the plunger.

In some embodiments, the first switch is a magnetic switch. The detector arrangement may further comprise a magnetic trigger point coupled to the movement of the plunger. The magnetic trigger point may be configured to actuate the first switch upon departure of the plunger from the first position.

This particular arrangement of magnets is an example of a detector arrangement that possesses the advantages of detecting the departure of the plunger, as discussed above. The first switch may correspond to the second magnet and the magnetic trigger point may correspond to the first magnet, as described below.

Alternatively, the first switch may be a mechanical switch, such as a micro switch. In such a case, the trigger point would be a mechanical trigger point (such as a flange, rib, protrusion, or similar) configured to actuate the first switch upon departure of the plunger from the first position.

In some embodiments, the detector arrangement further comprises a second switch located at the second position. The second switch may be configured to detect an arrival of the plunger at the second position.

It is contemplated that the detector arrangement may instead comprise the second switch, without the first switch being present.

In some embodiments, the second switch is a magnetic switch. The magnetic trigger point may be further configured to actuate the second switch upon arrival of the plunger at the second position.

Similarly to the first switch, the second switch may be a mechanical switch, such as a micro switch.

In some embodiments, the auto-injector further comprises a needle shroud at the front end of the auto-injector. The detector arrangement and/or the indicator arrangement may be configured to be activated responsive to the needle shroud being pressed against an injection site.

In such embodiments, the detector arrangement may comprise a shroud sensor as discussed herein.

In some embodiments, the detector arrangement and/or the indicator arrangement are deactivated responsive to the needle shroud being removed from the injection site.

Alternatively or additionally, the indicator arrangement may be configured to provide an alert indication if the needle shroud is removed from the injection site before the holding time is complete. Similarly, the completion indication may only be provided if the needle shroud has been pressed against the injection site during the entirety of the holding period.

In some embodiments, the auto-injector further comprises a viewing window. The viewing window may be configured to allow a user to view a visual marker coupled to the plunger of the syringe. The viewing window may be positioned such that the visual marker is visible when the plunger is in the second position.

In some embodiments, the auto-injector may further comprise a transmitter configured to transmit delivery information to a user device.

In some embodiments, the delivery information includes a delivery indication of whether the front end of the auto-injector was held against an injection site for the complete duration of the plunger being driven from the first position to the second position.

Advantageously, this informs a user as to whether the plunger completed a full delivery stroke while the auto-injector was held against the injection site. The user can take any remedial action required, such as seeking medical attention or delivering a further dose, if the full delivery stroke was not completed.

In some embodiments, the delivery indication further indicates whether the front end of the auto-injector was held against the injection site for the complete duration of the holding time.

Advantageously, this informs a user as to whether a complete and optimal dose of medicament was delivered. If the delivery indication indicates that the auto-injector was not held for the complete duration, the user may expect that the full dose was not delivered (because the bung of syringe had not expanded) or some medicament may leak from the injection site (because the medicament had not dispersed into the soft tissue).

The following features, characteristics, and advantages are also contemplated herein. According to an aspect, there is provided an auto-injector comprising a housing for receiving a syringe having a plunger, a needle, and a barrel containing a dose of medicament; a drive mechanism for driving the plunger from a first position to a second position to expel the dose from the barrel of the syringe through the needle at a front end of the auto-injector; a detector arrangement for detecting arrival of the plunger at the second position and for detecting removal of the needle from an injection site and for providing signals indicative of an occurrence time of each of the detected events.

The arrival of the plunger at the second position may act as an indication of the completion of the delivery stroke. Advantageously, the occurrence times of the arrival of the plunger and of the removal of the needle provide a proxy for whether a full-dose delivery has optimally and completely occurred. For example, the auto-injector may determine, whether a bung of the syringe has relaxed between the occurrence times. Alternatively or additionally, the auto-injector may determine whether the delivered medicament has dissipated under the injection site (e.g. has been absorbed by the soft tissue) between the occurrence times. The detected events refers to the detection of the arrival of the plunger at the second position and to the detection of the removal of the needle from the injection site.

The housing may comprise a hinged door openable to receive the syringe into the auto-injector. The syringe may comprise a safety syringe. The safety syringe may comprise a safety mechanism to protect a user from the needle. The safety syringe may comprise a needle sheath. The needle sheath may be configured to cover the needle after use of the syringe. Alternatively or additionally, the safety syringe may be configured to cause the needle to retract within the barrel of the syringe. It is noted that the term ‘needle’ is used herein as a general term that encompasses both pointed-tip needles and flat-tip needles (commonly referred to as ‘cannulas’).

The drive mechanism may comprise a drive spring configured to provide a force to drive the plunger from the first position to the second position. The drive spring may comprise a compression spring and/or a tension spring. The drive mechanism may comprise a plunger driver configured to abut the plunger and provide a force in order to drive the plunger.

The auto-injector may comprise a priming mechanism configured to prime the drive mechanism prior to operation of the release mechanism. The priming mechanism may be operated by movement of the door of the auto-injector.

The detector arrangement may be configured to measure an elapsed time from the arrival of the plunger at the second position, and to provide a signal indicative of the elapsed time being equal to or greater than a threshold time. Optionally, the detector arrangement may be configured to stop measuring the elapsed time at the occurrence time of the removal of the needle from the injection site.

Advantageously, the measurement of the elapsed time between the occurrence time of the arrival of the plunger and the occurrence time of the removal of the needle may provide a proxy for whether a full-dose delivery has optimally and completely occurred. For example, the auto-injector may determine, based on the elapsed time, whether a bung of the syringe has relaxed. Alternatively or additionally, the auto-injector may determine, based on the elapsed time, whether the delivered medicament has dissipated under the injection site (e.g. has been absorbed by the soft tissue).

The threshold time may be a typical period of time taken for a resilient syringe bung to relax and expand. A typical syringe which may be used with the auto-injector may comprise a resilient syringe bung that has well-known properties. For example, the resilient syringe bung may have a typical amount by which it is compressed during medicament delivery. The compression may be caused by the force and pressure applied by the drive mechanism. The resilient syringe bung may subsequently expand from its ‘compressed’ state into a ‘relaxed’ state. The expansion may take a typical period of time that is well-known. This period of time may be referred to herein as a ‘relaxation time’. It will be appreciated that it is not necessary for the exact relaxation time to be known. Instead, it is sufficient to know the relaxation time approximately. The threshold time may then be chosen to be equal to or slightly longer than the relaxation time. For example, the relaxation time may be 1, 2, 3, 4, 5, 6, or more seconds from after the end of the delivery stroke.

Advantageously, by comparing the elapsed time with a threshold time comprising the relaxation time, it can be determined whether or not the bung has expanded before or after the auto-injector is removed from the injection site. Since the expansion of the bung may result in more medicament being delivered from the syringe, this can be used to determine whether the full dose has been delivered or not.

The threshold time is optionally a typical period of time taken for a medicament to disperse into soft tissue.

After the delivery of medicament, the medicament may exist in the soft tissue that is immediately proximate to the needle. At this stage, the medicament may exist at a pressure high enough such that, if the needle were removed, the medicament would leak from the injection site. After a period of time, the medicament may disperse into, or be absorbed by, the surrounding soft tissue. This may result in a reduced pressure of the medicament such that leakage from the injection site would not occur. This period of time, referred to herein as ‘dispersion time’, may be well known. It will be appreciated that it is not necessary for the exact dispersion time to be known. Instead, it is sufficient to know the dispersion time approximately. The threshold time may then be chosen to be equal to or slightly longer than the dispersion time. For example, the dispersion time may be 1, 2, 3, 4, 5, 6, or more seconds from after the medicament has been delivered.

Advantageously, by comparing the elapsed time with a threshold time comprising the dispersion time, it can be determined whether or not the medicament has dispersed before or after the auto-injector is removed from the injection site. Since the medicament may leak if it has not sufficiently dispersed before the auto-injector is removed, this can be used to determine whether the medicament has or will have leaked from the injection site.

The threshold time may be defined by the greater of the relaxation time and the dispersion time, in order to ensure that both times have elapsed. For example, if the relaxation time is known to be 4 seconds, and the dispersion time is known to be 3 seconds, the threshold time may be chosen to be 4 or 5 seconds, such that it is equal to or greater than the relaxation time. The threshold time may also be chosen to account for any uncertainty in either of these times. For example, if the relaxation time is approximately 4 seconds, the threshold time may be chosen to be 5 or 6 seconds, so that the relaxation time is likely to have passed by the expiry of the threshold time.

The detector arrangement may be configured to transmit the signals to an indicator arrangement, wherein the indicator arrangement is configured to provide an indication to a user based on a received signal. Advantageously, this allows a user to be notified of the status of delivery, with respect to how long the auto-injector was held against the injection site. The indication may notify the user that the plunger has arrived at the second position. The indication may notify the user that the needle has been removed from the injection site. The indication may notify the user of the duration of the elapsed time. Alternatively or in addition, the indication may notify the user of the relationship between the elapsed time and the threshold time, e.g. that the elapsed time is longer, shorter, or equal to the threshold time.

The signals may be an alert signal configured to cause the indicator arrangement to provide an alert indication that the needle has been removed from the injection site before the expiry of the threshold time. Advantageously, this alerts the user to the fact that the delivery of the medicament was sub-optimal and/or incomplete. The user then may take any necessary action to combat negative effects. For example, with reference to the relaxation time of the bung, the indication may notify the user that slightly less than the full dose was delivered. As a further example, with reference to the dispersion time of the medicament, the indication may notify the user that the medicament may leak from the injection site.

The signals may be a completion signal configured to cause the indicator arrangement to provide a completion indication that the elapsed time is equal to or greater than the threshold time. Advantageously, this notifies the user to the fact that the delivery of the medicament was optimal and/or complete. More specifically, this notifies the user that the needle of the auto-injector may be removed with little to no risk of leakage of the medicament (either from the injection site, due to the medicament pressure, or from the needle, due to the bung expansion). The user may then remove the auto-injector from the injection site with the knowledge that the delivery is complete.

The indicator arrangement may be further configured to provide a progress indication to the user while the plunger is being driven to deliver medicament. More specifically, the indicator may provide the progress indication between the departure of the plunger from the first position and the arrival of the plunger at the second position. Additionally, the indicator may continue to provide the progress indication between the arrival of the plunger at the second position and either the removal of the needle from the injection site, or the elapsed time exceeding the threshold time. Alternatively, the indicator may provide a different indication once the plunger has arrived at the second position. For example, the different indication may indicate that the plunger movement is complete but that the auto-injector should continue to be held in place until the threshold time has elapsed.

The indicator arrangement may be configured to provide a visual, audible, and/or tactile indication. For example, the indicator arrangement may comprise an illumination device. For example, the indicator arrangement may comprise one or more light emitting diodes (LEDs) and/or a display screen. In such cases, and when the indicator arrangement is located on the auto-injector itself, the housing may comprise a viewing window through which the illumination device can be viewed. The indicator arrangement may comprise a sound emitter. The indicator arrangement may comprise a haptic feedback device.

The indicator arrangement may be located on the auto-injector, and wherein the detector arrangement is configured to transmit the signal to the indicator arrangement as an electrical signal.

Optionally, the indicator arrangement is a user device separate from the auto-injector, wherein the auto-injector further comprises a transmitter, and wherein detector arrangement is configured to transmit, via the transmitter, the signal to the indicator arrangement using a short-range transmission protocol.

The user device may comprise a handheld device. The user device may comprise a dedicated indication device for use with the auto-injector. Alternatively, the user device may comprise a mobile phone. In this case, the mobile phone may use a dedicated application for monitoring and/or interfacing with the auto-injector. The short-range transmission protocol may comprise Bluetooth®.

The detector arrangement may detect departure of the plunger from the first position. One or more switches of the detector arrangement may be configured (as described below) to be actuated upon departure of the plunger from the first position. The detector arrangement may be configured to detect arrival or departure of the plunger from various positions during the delivery. For example, the detector arrangement may be configured to detect arrival or departure of the plunger from the first position, the second position, and/or one or more intermediate positions. References to detecting the position of the plunger may instead refer to detecting the position of the plunger driver, which naturally acts as a proxy to the plunger position.

The auto-injector may have a needle shroud at the front end of the auto-injector, wherein the needle shroud is configured to move forwardly into an extended position upon removal of the front end from the injection site.

The needle shroud may be forwardly biased by a shroud spring. The needle shroud may be configured to lock in the extended position to prevent access to the needle after removal of the front end from the injection site. The needle shroud may be biased forwardly into the extended position prior to operation of the drive mechanism, priming mechanism, and/or the release mechanism. The needle shroud may be configured to move rearwardly when the front end is pressed against the injection site. The needle shroud may be configured to move rearwardly prior to injection and may be configured to lock after injection. Rearward movement of the needle shroud may expose the needle and cause the needle to puncture the injection site. Rearward movement of the needle shroud may operate the release mechanism, thereby causing the drive mechanism to drive the plunger.

The detector arrangement may include a shroud sensor configured to sense a position of the needle shroud, wherein the detector arrangement detects the removal of the needle from the injection site responsive to the shroud sensor sensing that the needle shroud has moved forwardly into the extended position.

The detector arrangement may use the position of the needle shroud as a proxy for the position of the needle relative to the injection site. More specifically, the detector arrangement may use the position of the needle shroud as a proxy for whether the needle is embedded within, or removed from, the injection site. Advantageously, this allows a determination of whether or not an injection is ongoing or has been terminated. Advantageously, this reduces the need for other components on the auto-injector in order to sense whether the auto-injector is being held against the injection site. In addition, this may reduce the manufacturing complexity and cost of the auto-injector.

To this end, references herein to removal of the needle from the injection site, or similar, may interchangeably refer to movement of the needle shroud into the extended position, and vice versa. The extended position may correspond to full extension of the needle shroud from the front end of the auto-injector. Alternatively, the extended position may correspond to an extension of the needle shroud corresponding to the length of the needle.

The shroud sensor may be a switch. The shroud sensor may be a mechanical switch, a micro-switch, an electrical switch, a magnetic switch, and/or any other suitable switch known to the skilled person.

The detector arrangement may comprise one or more switches. The one or more switches may comprise mechanical switches, micro-switches, electrical switches, magnetic switches, and/or any other suitable switches known to the skilled person. The one or more switches may be configured to be actuated upon arrival of the plunger at the second position.

The detector arrangement may comprise a trigger point coupled to the movement of the plunger. For example, the trigger point may be located on the plunger. Alternatively, the trigger point may be located on the plunger driver. The detector arrangement may comprise a switch coupled to the housing. The trigger point may be configured to actuate the switch upon relative movement between the trigger point and the switch. For example, the trigger point may comprise a first magnet and the switch may comprise a second magnet, the first magnet being configured to interact with the second magnet as the first magnet moves past the second magnet. Alternatively, the trigger point may comprise a rib and the switch may comprise a mechanical switch, the rib being configured to operate the switch as the rib moves past the switch.

The detector arrangement may comprise a proximity sensor for sensing the proximity of the front end to the injection site. The proximity sensor may be a mechanical sensor and/or an optical sensor. The proximity sensor may detect whether the needle is inserted into the injection site.

The auto-injector may include a release mechanism that, upon actuation, causes the drive mechanism to drive the plunger, wherein the release mechanism is configured to only allow actuation if the detector arrangement detects that the front end is in contact with the injection site.

For example, the release mechanism may be a trigger button. In this case, the trigger button may be in a locked state until the detector arrangement detects that the needle is embedded in the injection site (e.g. until it detects that the needle shroud is moved rearwardly by being pressed against the injection site). Alternatively, the release mechanism may be the needle shroud. In this case, the needle shroud is moved rearwardly by pressing the front end against the injection site, thereby causing the drive mechanism to drive the plunger.

The auto-injector may also include a syringe having a plunger, a needle, and a barrel. The syringe may include a resilient bung. The auto-injector may further comprise a power supply to provide power to the detector arrangement, the indicator arrangement, and/or the drive mechanism.

According to an aspect, there is provided a system comprising an auto-injector as described herein; and a user device configured to receive one or more indication signals using a short-range transmission protocol, and further configured to, responsive to receiving one or more indication signals, provide an indication corresponding to the received signal.

### BRIEF DESCRIPTION OF THE DRAWINGS

Exemplary embodiments will now be described with reference to the Figures, in which:

FIG. 1 is a front view of an auto-injector containing a syringe, with a plunger in a first position;

FIG. 2A is a enlarged interior perspective view of the auto-injector of FIG. 1, with the plunger in the first position;

FIG. 2B is a enlarged interior perspective view of the auto-injector of FIG. 1, with the plunger in a second position;

FIG. 3 is a front view of the auto-injector of FIG. 1, with the plunger in the second position;

FIG. 4 is an enlarged front view of an indicator arrangement of the auto-injector of FIG. 1;

FIGS. 5A-5B are enlarged front views of the indicator arrangement of FIG. 4 during a cycle of indication; and

FIGS. 6A and 6B are enlarged front views of the indicator arrangement of FIG. 4 during a completion indication.

### DETAILED DESCRIPTION

Generally disclosed herein are exemplary apparatus for auto-injectors. The term “auto-injector” is used herein to refer to a device configured to receive a syringe and provide mechanical assistance to the user during delivery of medicament from the syringe. The auto-injectors may be configured to receive and operate a standard syringe (i.e. not a safety syringe) and/or a safety syringe.

In the following examples, the terms “forward” and “front” refer to the end of the auto-injector, or component thereof, that faces the injection site. The injection site will typically be a patient's skin. In other words, the front end of the auto-injector is the end proximal to the injection site during use. Likewise, the term “rear” refers to the non-injection-site end of the auto-injector, or component thereof. In other words, the term “rear” means distal from the injection site during use. Further, the term longitudinal is used to encompass a direction along or parallel to a longitudinal axis, running from the rear to the front, of the injection device.

Features of the exemplary arrangements disclosed herein are described as being “coupled” to other features. This term encompasses any coupling that results in coupled features moving together in any direction. The term “coupled” also encompasses any one of a connection between features, an abutment of one feature against another, and an engagement of one feature with another, and such coupling may be direct or may be indirect, i.e. with a third feature therebetween.

In general terms, the disclosure is directed to improving the delivery of a medicament from an auto-injector by ensuring an optimal and complete delivery. By ‘optimal and complete’ it is meant that substantially all of the medicament present in the syringe is delivered to and absorbed by the injection site. It is an appreciation of the present Applicant that leakage can occur both from the injection site and from the needle once the auto-injector has been removed from the injection site, thereby resulting in sub-optimal or incomplete medicament delivery. The Applicant has appreciated that this leakage may occur through two separate mechanisms.

Firstly, a plunger of the syringe typically comprises a bung that is made of a resilient material, for example rubber. The bung is typically somewhat compressed during medicament delivery, and will subsequently ‘relax’ and expand after the plunger has completed its motion through the barrel. During this period of expansion, a small amount of medicament may be expelled from the syringe. If the auto-injector, or specifically the syringe needle, is removed from the injection site prematurely, this relaxation and expansion may lead to medicament being expelled outside of the injection site, leading to an incomplete dosage of medicament being delivered.

Secondly, after delivery, the medicament can be momentarily at a high pressure under the skin of the user. Over a short period of time, the medicament will dissipate into the surrounding soft tissue, leading to a reduction of the pressure. If the auto-injector, or specifically the syringe needle, is removed from the injection site prematurely, the medicament will not have yet dissipated and the high pressure can lead to some of the medicament leaking back out of the injection site.

Exemplary auto-injectors disclosed herein provide signals indicative of the completion of the delivery stroke, and of the removal of the needle from the injection site. By providing these signals, it can be ensured that sufficient time is allowed for the bung to relax and/or for the medicament to disperse under the injection site before the needle is removed from the injection site, thereby reducing or effectively preventing the aforementioned leakage. The exemplary auto-injectors disclosed herein may also measure the time after the plunger has completed its delivery stroke, and optionally provide an indication to the user. The time may be measured until the auto-injector detects that the needle has been removed from the injection site.

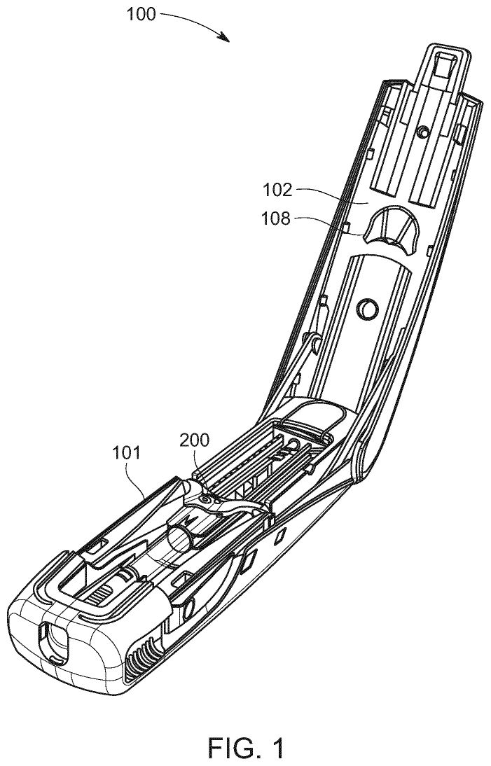

FIG. 1 depicts an exemplary auto-injector 100 that has a syringe 200 fitted within a housing of the auto-injector 100. The auto-injector 100 is depicted prior to delivery of medicament from the syringe 200. The housing of the auto-injector 100 comprises a main body 101 and a door 102, which may be hinged relative to the main body 101. The door 102 is operable between an open position, in which the syringe 200 can be inserted, and a closed position. FIG. 1 depicts the door 102 in the open position.

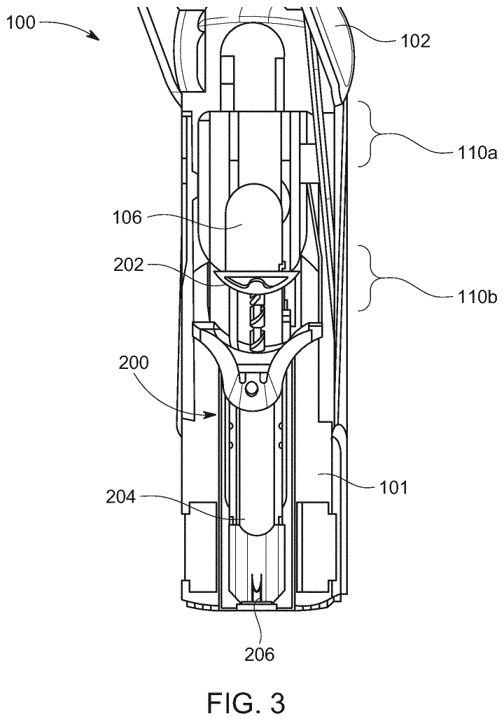

The auto-injector 100 comprises a viewing window 118 and an indicator arrangement 120. The functionality of the indicator arrangement 120 will be explained in greater detail with reference to FIGS. 4-6B. In FIG. 1, the viewing window 118 and the indicator arrangement 120 are depicted as being located on the door 102, but it will be understood that these components could be located elsewhere on the auto-injector housing. The indicator arrangement 120 may instead not be located on the auto-injector 100 at all, but instead be provided by a separate device. For example, the indicator arrangement 120 may comprise a separate user device configured to communicate with the auto-injector 100. In the Figures, the indicator arrangement 120 is depicted as an arrangement of four LEDs arranged in a circle. It will understood that other configurations of LEDs are possible. Furthermore, other indicator arrangements are contemplated herein, for example, audio or haptic indicators, or visual indicators other than LEDs.

The viewing window 118 allows a user to view inside the housing of the auto-injector 100. The specific purpose of the viewing window 118 will be described in detail below with respect to the injection process.

The syringe 200 fitted into the auto-injector may be a safety syringe, as shown in the Figures. The syringe 200 comprises a plunger 202 for driving medicament from a barrel 204 and out of a needle 206. The forward end of the plunger 202 comprises a bung 208. The bung 208 is typically made of a resilient material. The bung 208 refers specifically to the portion of the plunger 202 that makes direct contact with the medicament contained within the barrel 204.

The auto-injector 100 also comprises a needle shroud 108, or simply ‘shroud’ 108. The shroud 108 is not shown in FIG. 1. When the cap is removed from the auto-injector the shroud 108 extends from the front end of the auto-injector 100. The shroud 108 is forwardly biased by a spring (not shown). Therefore, in its present state (i.e. prior to medicament delivery), the shroud 108 protrudes from the front end of the auto-injector 100, but may be pressed rearwardly (i.e. against the bias of the spring) in order to expose the needle 206. For example, the shroud 108 may be pressed rearwardly when the front end of the auto-injector 100 is pressed against the injection site, in order to insert the needle 206 into the injection site. The auto-injector further comprises a shroud sensor 116 that detects the position of the shroud 108 relative to the housing of the auto-injector (e.g. whether the shroud 108 is in an extended position).

The auto-injector 100 comprises a drive mechanism 104 for driving the plunger 202 from a first position 110a to a second position 110b. The first position 110a may be considered to be an initial position, and may refer to the position of the plunger 202 when it is fully removed from the barrel 204 such that no medicament has been delivered from the needle 206. The second position 110b may be considered to be a final position, and may refer to the position of the plunger 202 when it is fully inserted into the barrel 204 such that the full dosage of medicament has been delivered from the needle 206. In the Figures, the first position 110a and the second position 110b are labelled as respective positions of the plunger head (i.e. the distal end of the plunger). This is for illustrative purposes only and it is to be understood that the first and second positions refer generally to the positions of the plunger, and not necessarily to a particular portion of the plunger.

The drive mechanism 104 typically comprises a drive spring 105 and a plunger driver 106. The drive spring 105 provides a forward bias against the plunger driver 106, which is coupled to the plunger 202. Upon actuation of the auto-injector 100, the drive spring 105 drives the plunger driver 106 forwards, thereby driving the plunger 202, leading to medicament delivery. It will be understood by the skilled person that many different forms of drive mechanism are known for auto-injectors, and that the present disclosure may be applicable to auto-injectors with different forms of the drive mechanism 104.

The auto-injector 100 also comprises a release mechanism that, upon operation, causes the drive mechanism 104 to drive the plunger 202. As shown in the Figures, the release mechanism may be operated by the shroud 208. That is, upon depression of the shroud 208, the drive mechanism 104 is released in order to drive the plunger 202. In such cases, after fitting of the syringe 200 into the auto-injector 100, the user simply presses the front end of the auto-injector 100 against the injection site (thereby moving the shroud 208 rearwardly) and the injection process will commence. It will be understood that alternative forms of the release mechanism could be provided, for example, by way of a trigger button on the rear end of the auto-injector 100.

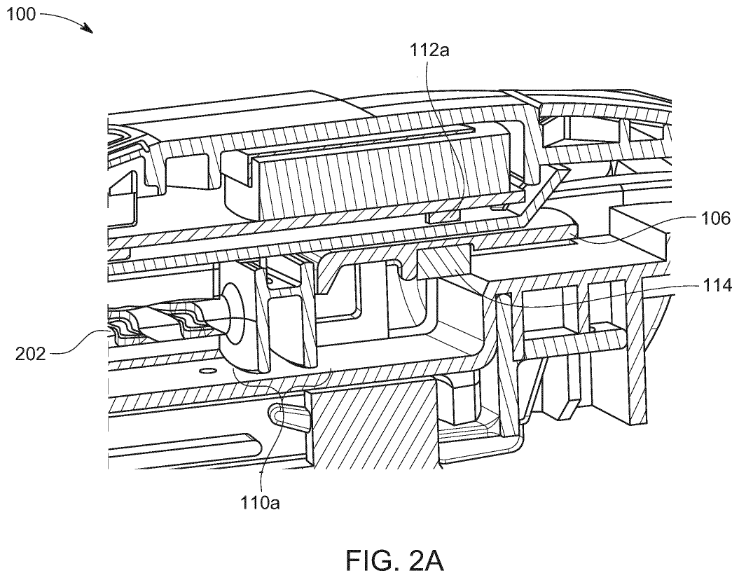

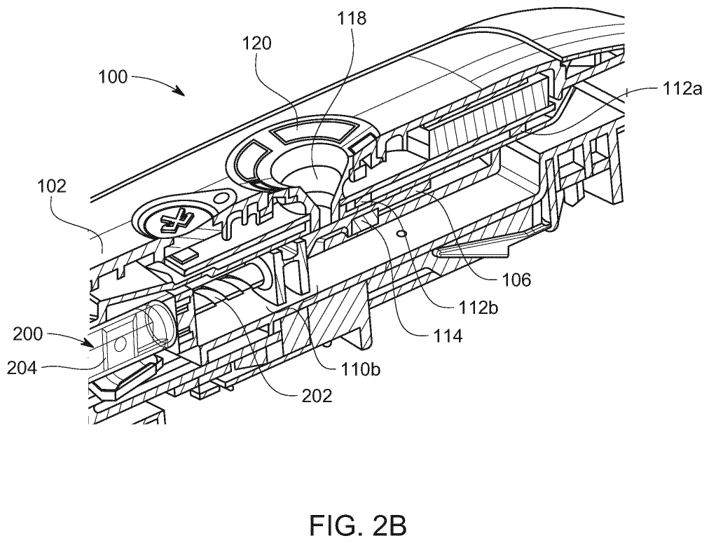

The injection process will now be described with reference to FIGS. 2A-3.

FIGS. 2A and 2B show the plunger driver 106 abutting the plunger 202. From these views, it can be seen that the auto-injector 100 further comprises a detector arrangement comprising a first switch 112a and a second switch 112b. In this example, the first switch 112a and the second switch 112b are magnetic switches, however, it will be understood that other forms of switches may be used.

Plunger driver 106 comprises a trigger point 114. The trigger point 114 is configured to interact with the first switch 112a and the second switch 112b as the trigger point 114 passes the respective switch. In this case, this means that trigger point 114 is a magnet that interacts with the switches. It will be understood that trigger point 114 could take a different form depending on the form of the switches. For example, trigger point 114 could be a protruding rib in the case of mechanical switches. Since the trigger point 114 is located on the plunger driver 106, the trigger point 114 is coupled to the plunger driver 106 and the plunger 202. In alternative arrangements, the trigger point 114 could be located directly on the plunger 202 itself.

Starting with FIG. 2A, the plunger 202 is shown in a first position 110a. In this position, the plunger driver 106 is located such that the trigger point 114 is aligned with first switch 112a. In this position, the plunger 202 is fully extended from the barrel 204. In other words, this shows the position of the components prior to delivery of the medicament. Upon operation of the release mechanism, the plunger driver 106 begins to move forwardly, driving the plunger 202 into the barrel 204. The trigger point 114 will depart from the first switch 112a, thereby triggering the first switch 112a. This indicates the beginning of the delivery sequence, or delivery stroke. In response to the triggering of the first switch 112a, the detector arrangement may transmit a signal to the indicator arrangement 120, such that the indicator arrangement 120 provides an indication that the delivery sequence has started.

The plunger driver 106, and the plunger 202, continue to move forwardly along the auto-injector 100. During this time, the indicator arrangement 120 may provide an indication that the delivery sequence is ongoing.

Turning to FIG. 2A, the plunger 202 has progressed to a second position 110b, which may correspond to a full-dose delivery of medicament from the barrel 204. In other words, the second position 110b may correspond to the bung 208 being at the front end of the barrel 204. Upon arrival of the plunger 202 at the second position 110b, the trigger point 114 is aligned with the second switch 112b. The arrival of the trigger point 114 at the second switch 112b triggers the second switch 112b. This indicates the end of the delivery sequence, or delivery stroke. In response to the triggering of the second switch 112b, the detector arrangement may transmit a signal to the indicator arrangement 120, such that the indicator arrangement 120 provides an indication that the delivery sequence has completed. Alternatively, the indicator arrangement 120 may continue to provide an indication that the delivery sequence is ongoing (since the user should not yet remove the auto-injector 100 from the injection site).

Alternatively or in addition, the plunger driver 106 may act as a visual indicator to the user that the delivery sequence is complete. As seen in FIG. 3, the plunger driver 106, upon arrival at the second switch 112b, is aligned with the viewing window 118. The visible presence of the plunger driver 106 in the viewing window 118 acts as an indication that the plunger 202 has progressed to the second position 110b. The plunger driver 106 may comprise a visual indicator (e.g. a marking) to aid its visibility through the viewing window 118.

Upon arrival of the plunger 202 at the second position 110b (i.e. once the second switch 112b has been triggered), the detector arrangement may provide a signal (e.g. by transmitting a signal to the indicator arrangement) indicating the arrival of the plunger 202 at the second position 110b. The detector arrangement may also begin measuring an elapsed time. The elapsed time indicates how long has passed since the delivery stroke was completed.

The detector arrangement may continue to measure the elapsed time until the shroud sensor 116 detects that the shroud 108 has extended. Upon extension of the shroud 108, the detector arrangement may provide a signal (e.g. by transmitting a signal to the indicator arrangement) indicating the removal of the needle 206 from the injection site. While the auto-injector 100 is being held against the injection site, the shroud 108 will be depressed into the auto-injector 100, as seen in FIG. 3. Since the extension of the shroud 108 corresponds to the removal of the needle 206 from the injection site, the elapsed time will correspond to the total time that the auto-injector 100 was held against the injection site after delivery is complete. The elapsed time may be indicated to the user, for example by the indicator arrangement 120, so that the user knows for how long they held the auto-injector 100 in place after delivery was complete.

The detector arrangement may compare the elapsed time against a threshold time. The indicator arrangement 120 may provide an indication to the user based on the comparison of the elapsed time against the threshold time. For example, the indicator arrangement 120 may provide an indication that the elapsed time is less than, equal to, or greater than the threshold time.

The threshold time may be chosen based on the period of time it takes for the bung 208 to relax and expand after the delivery stroke is complete. The period of time may be referred to as the ‘relaxation time’. The threshold time may be chosen based on the period of time it takes for the delivered medicament to disperse into the soft tissue. The period of time may be referred to as the ‘dispersion time’. The relaxation time and/or the dispersion time may be values that are well-known or that can be calculated prior to the manufacture of the auto-injector 100. The threshold time may be chosen to be the longer of the relaxation time and the dispersion time. The threshold time may be, for example, 5 seconds.

Once the second switch 112b is triggered, the detector arrangement begins measuring the elapsed time and comparing it with the threshold time. The indicator arrangement 120 may continue to provide an indication that the delivery is ongoing while the elapsed time is less than the threshold time. If the shroud sensor 116 detects that the shroud 108 has moved into the extended position (i.e. the user has removed the auto-injector 100 from the injection site) while the elapsed time is less than the threshold time, the indicator arrangement 120 provides an alert indication to indicate to the user that the auto-injector 100 was removed too early. Providing the auto-injector 100 is not removed too early, the indicator arrangement 120 may provide a completion indication to the user when the elapsed time equals the threshold time. This indicates to the user that the threshold time has been reached and the auto-injector 100 can be removed from the injection site. Once the shroud sensor 116 detects that the shroud 108 has moved into the extended position, the detector arrangement may cease measuring the elapsed time.

After the auto-injector 100 is removed from the injection site, as described above, by way of its forward bias, the shroud 108 moves into the extended position. In contrast to the extended position of the shroud 108 described with reference to FIG. 1, the shroud 108 may be locked in the extended position after delivery, such that the needle 206 can no longer be accessed. In other words, when in the extended position prior to delivery, the shroud 108 may be pressed against its bias (e.g. to begin the delivery sequence), but when in the extended position after delivery, the shroud 108 cannot be pressed against its bias.

The auto-injector 100 may be reset and reused by opening the door 102, removing the syringe 200, replacing with a new syringe, and repeating the above process.

Examples of indications that may be displayed by the indicator arrangement 120 will now be described with reference to FIGS. 4-6B.

FIG. 4 depicts a close-up view of the indicator arrangement 120. The indicator arrangement 120 comprises four electrically-operated display elements 120a-d (or, simply, ‘display elements 120a-d’) which, in this example, are arranged as four quadrants in a circular arrangement. In the centre of the circular arrangement is the viewing window 118 that allows a user to view inside the housing of the auto-injector 100, as described above. It will be appreciated that other arrangements of the electrically-operated display elements 120a-d are possible. In this example, the electrically-operated display elements 120a-d are provided by four LEDs, but other elements (such as a display screen or LCD elements) may be used.

The indicator arrangement 120 further comprises one or more (in this case, two) auxiliary indicators 121a, 121b. The auxiliary indicators are configured to provide an indication of one or more states of the auto-injector that are unrelated to the delivery sequence. In the depicted embodiment, auxiliary indicator 121b is configured to provide indications based on a Bluetooth connectivity status of the auto-injector 100. For example, the auxiliary indicator 121b may indicate whether the auto-injector 100 is connected to, connecting to, disconnecting from, or disconnected from a user device via Bluetooth. The auxiliary indicator 121a may be configured to indicate another state of the auto-injector 100, such as power status, battery status, and/or maintenance status.

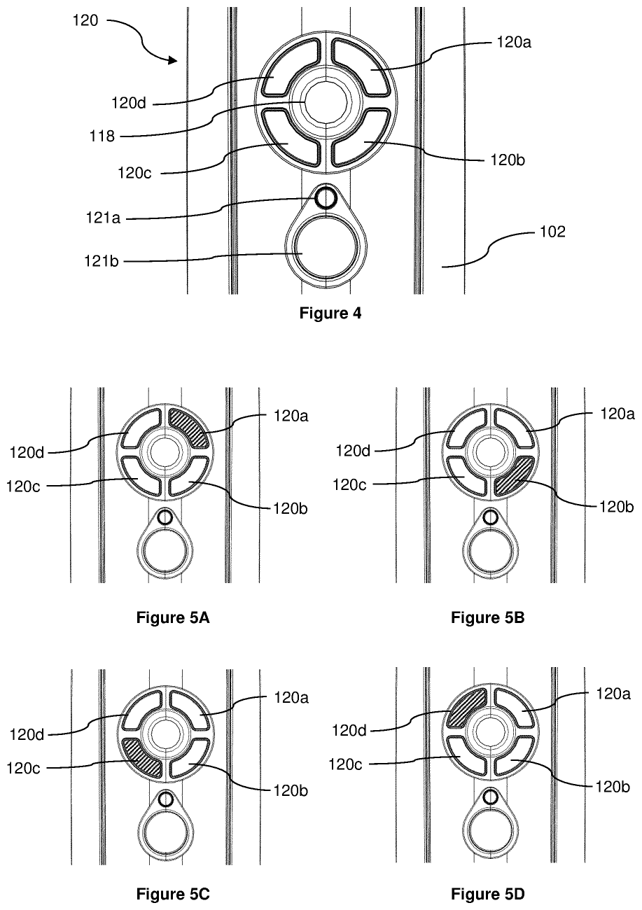

An exemplary visual indication that the indicator arrangement 120 is configured to provide is depicted in FIGS. 5A-5D. This visual indication may be referred to herein as a progress indication. The visual indication may commence responsive to the detector arrangement detecting the departure of the plunger 202 from the first position 110a. Specifically, the visual indication may commence at the beginning of the delivery sequence, once the trigger point 114 departs from the first switch 112a, thereby triggering the first switch 112a.

In FIG. 5A, the visual indication begins with display element 120a activated (e.g. illuminated). This is depicted in the Figures as diagonal hatching. The visual indication progresses through FIGS. 5B-5D with each display element 120b-d operated sequentially such that the visual indication gives the appearance of rotation through the display elements 120a-d. The process depicted in FIGS. 5A-5D represents a single cycle of indication. It will be appreciated that, while the cycle is depicted as starting with display element 120a being activated first, any of the display elements 120a-d may instead be activated first.

The cycle may be repeated continuously during the delivery sequence, until the detector arrangement detects the arrival of the plunger 202 at the second position 110b or, more specifically, until the arrival of the trigger point 114 at the second switch 112b triggers the second switch 112b. The cycle of indication may continue to be repeated for a holding period after the arrival of the plunger 202 at the second position 110b (e.g. until the elapsed time has reached the threshold time). Advantageously, this informs the user that the auto-injector 100 should not yet be removed from the injection site.

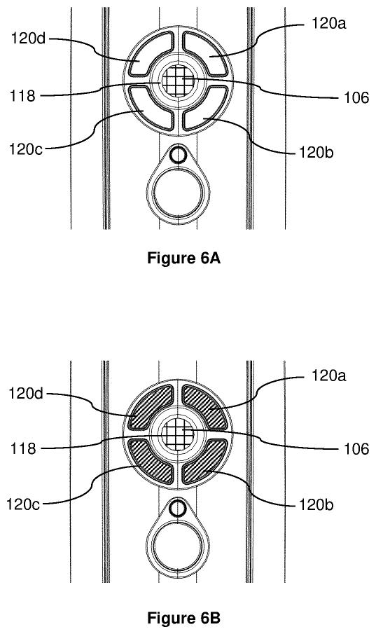

Once the holding period has expired (e.g. the elapsed time is equal to or greater than the threshold time), the indicator arrangement 120 may provide a completion indication, as shown in FIGS. 6A-6B. As shown in FIGS. 6A and 6B, the plunger 202 has reached the second position 110b and so the plunger driver 106 (depicted as cross-hatching) is visible through viewing window 118. This provides an indication to the user that the delivery sequence is complete. Advantageously, this indication is independent of the indicator arrangement 120 and so can still be used if the indicator arrangement 120 is unpowered or damaged.

FIGS. 6A and 6B depict example forms of a completion indication that may be provided by the indicator arrangement 120.

FIG. 6A depicts an indication in which all four display elements 120a-d are simultaneously in a deactivated (e.g. unilluminated) state. FIG. 6B depicts an indication in which all four display elements 120a-d are simultaneously in an activated (e.g. illuminated) state. The completion indication may comprise either of these two indications. In other words, the completion indication may comprise simply switching off or switching on all four display elements 120a-d. Alternatively, the completion indication may comprise flashing of the display elements 120a-d. In such a case, the process in FIGS. 6A-6B represent a single cycle of indication, and the completion indication can be understood to be a repetition of the single cycle. The cycle may be repeated (i.e. the display elements may be flashed) several times (e.g. 2, 3, 4, 5 or more times). Advantageously, the completion indication informs the user that the auto-injector 100 can now be removed from the injection site.

1. An auto-injector comprising:
a housing for receiving a syringe having a plunger, a needle, and a barrel containing a dose of medicament;
a drive mechanism configured to, when triggered by a user, automatically drive the plunger from a first position to a second position to expel the dose from the barrel of the syringe through the needle at a front end of the auto-injector;
a detector arrangement configured to detect a state of the plunger including at least departure from the first position and arrival at the second position; and
an indicator arrangement comprising a display having one or more electrically-operated display elements and configured to provide, responsive to the detector arrangement detecting the departure of the plunger from the first position, a visual indication comprising cycles of indications, the cycles of indications repeating until the detector arrangement detects the arrival of the plunger at the second position.

2. The auto-injector according to claim 1, wherein the cycles of indications present a sequence that moves in a continuous or repeated direction.

3. The auto-injector according to claim 1, wherein the one or more electrically-operated display elements are arranged in a circular arrangement.

4. The auto-injector according to claim 3, wherein the one or more electrically-operated display elements are configured to be operated sequentially to give the appearance of rotation through the electrically-operated display elements.

5. The auto-injector according to claim 1, wherein the one or more electrically-operated display elements are arranged in a linear arrangement.

6. The auto-injector according to claim 5, wherein the one or more electrically-operated display elements are configured to be operated sequentially to give the appearance of scrolling through the electrically-operated display elements.

7. The auto-injector according to claim 1, wherein the indicator arrangement is further configured to continue repeating the cycles of indication during a holding period that starts from when the detector arrangement detects the arrival of the plunger at the second position.

8. The auto-injector of claim 7, wherein the indicator arrangement is further configured to provide, responsive to the expiry of the holding period, a completion indication notifying the user that the auto-injector can be removed from the injection site.

9. The auto-injector of claim 7, wherein the holding time is greater than or equal to a typical period of time taken for a resilient syringe bung to relax and expand.

10. The auto-injector according to claim 7, wherein the holding time is greater than or equal to a typical period of time taken for a medicament to disperse into soft tissue.

11. The auto-injector according to claim 1, wherein the detector arrangement comprises a first switch located at the first position and configured to detect a departure of the plunger from the first position.

12. The auto-injector of claim 11, wherein the first switch is a magnetic switch and wherein the detector arrangement further comprises a magnetic trigger point coupled to the movement of the plunger, the magnetic trigger point being configured to actuate the first switch upon departure of the plunger from the first position.

13. The auto-injector of claim 11, wherein the detector arrangement further comprises a second switch located at the second position and configured to detect an arrival of the plunger at the second position.

14. The auto-injector of claim 12, wherein the detector arrangement further comprises a second switch located at the second position and configured to detect an arrival of the plunger at the second position and the second switch is a magnetic switch and wherein the magnetic trigger point is further configured to actuate the second switch upon arrival of the plunger at the second position.

15. The auto-injector of claim 1, further comprising a needle shroud at the front end of the auto-injector, wherein the detector arrangement and/or the indicator arrangement are configured to be activated responsive to the needle shroud being pressed against an injection site.

16. The auto-injector of claim 15, wherein the detector arrangement and/or the indicator arrangement are deactivated responsive to the needle shroud being removed from the injection site.

17. The auto-injector of claim 1, wherein the auto-injector further comprises a viewing window configured to allow a user to view a visual marker coupled to the plunger of the syringe, the viewing window being positioned such that the visual marker is visible when the plunger is in the second position.

18. The auto-injector of claim 1, further comprising a transmitter configured to transmit delivery information to a user device.

19. The auto-injector of claim 18, wherein delivery information includes a delivery indication of whether the front end of the auto-injector was held against an injection site for the complete duration of the plunger being driven from the first position to the second position.

20. The auto-injector of claim 19, wherein the delivery indication further indicates whether the front end of the auto-injector was held against the injection site for the complete duration of the holding time.

21. A system, comprising an auto-injector and a user device,
the auto-injector comprising:
a housing for receiving a syringe having a plunger, a needle, and a barrel containing a dose of medicament;
a drive mechanism configured to, when triggered by a user, automatically drive the plunger from a first position to a second position to expel the dose from the barrel of the syringe through the needle at a front end of the auto-injector;
a detector arrangement configured to detect a state of the plunger including at least departure from the first position and arrival at the second position and configured to transmit, via a transmitter, signals indicative of said states to an indicator arrangement provided by a user device using a short-range transmission protocol; and
the user device comprising
an indicator arrangement, the indicator arrangement comprising a display having one or more electrically-operated display elements and configured to provide, responsive to receiving a signal from the detector arrangement indicative of the plunger having departed from the first position, a visual indication comprising cycles of indications, the cycles of indications at least repeating until the indicator arrangement receives a further signal from the detector arrangement indicative of the plunger having arrived at the second position.
